(19) United States
(12) Patent Application Publication
(10) Pub. No.: US 2003/0166518 A1
(43) Pub. Date: Sep. 4, 2003

(54) Method for allergen characterization

(76) Inventors: Thomas A. Beardslee, Lincoln, NE (US); Michael G. Zeece, Lincoln, NE (US); Guatam Sarath, Lincoln, NE (US); John P. Markwell, Lincoln, NE (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 09/759,967

(22) Filed: Jan. 12, 2001

Related U.S. Application Data
(60) Provisional application No. 60/175,948, filed on Jan. 13, 2000.
(60) Provisional application No. 60/186,724, filed on Mar. 3, 2000.

(51) Int. Cl. A61K 38/00; A61K 39/395

(52) U.S. Cl. 514/12; 435/7.2; 424/131.1

(57) ABSTRACT

Disclosed are methods for the characterization of allergens using recombinant fusion protein in an enzyme-linked immunosorbent (ELISA) assay. Also disclosed are methods for determining allergen sensitivity and methods for immunotherapy using the ELISA of the present invention.

### CROSS REFERENCE TO RELATED APPLICATIONS

This application claims the benefit of provisional application No. 60/175,948, filed Jan. 13, 2000, which is hereby incorporated by reference in its entirety for all purposes.

### BACKGROUND

Type I allergy is a genetically determined hypersensitivity disease that affects approximately 20% of the population. Type I allergy is characterized by the production of immunoglobulin E (IgE) antibodies against what are normally harmless antigens (allergens). Immunoglobulin E is the least abundant class of immunoglobulins present in blood and other body fluids such as tears, nasal and bronchial secretions. Acute symptoms of Type I allergy include allergic rhinitis, conjunctivitis, asthma, dermatitis, and anaphylactic shock. These symptoms are due to the release of biological mediators such as histamine and leukotrienes from effector cells (mast cells and basophils). Release of biological mediators is due, in turn, to allergen induced crosslinking of Fc_{ε}RI-bound IgE antibodies bound to effector cells.

Diagnosis of Type I allergy can be accomplished by detection of allergen specific IgE in serum (Wide et al., Lancet, 2:1105-1107, 1967; Eriksson and Ahlstedt, Int. Arch. Allergy Appl. Immunol. 54:88-95, 1977). These tests, however, have not been found to correlate highly with clinical symptoms and usually must be confirmed by provocation testing. The low correlation with clinical allergies may be due to impurities in the allergens used in such tests resulting in false positives, or the masking of IgE binding sites when the allergen is bound to a solid surface such as occurs in ELISA assays or immunoblotting.

At present two methods of causative treatment for Type I allergy exist. The first is avoidance, in which the effected individual attempts to avoid the allergen. This method has obvious limitations due to the ubiquitous nature of many allergens. A second method is immunotherapy. In immunotherapy, the individual is administered increasing doses of the allergen in order to induce specific unresponsiveness. Immunotherapy is thought to act, at least in part, by stimulating an immunoglobulin G mediated immune response against the allergen. Then, when the individual is exposed to the allergen, binding of IgG to the allergen results in clearance of the allergen and inhibition of an IgE mediated allergic response.

There are two major disadvantages to allergen-specific immunotherapy using natural allergen extracts as is the common practice. First, the allergen extracts used consist of mixtures of allergenic and non-allergenic moieties which are difficult to standardize. Thus, it is possible that the allergen to which the individual reacts is not present in the mixture or that the individual will develop new sensitivities to components in the extracts during the course of treatment. A second, and potentially life-threatening problem, is that since the preparations contain biologically active allergens, administration may result in anaphylactic side effects.

Recombinant protein technologies have provided a means by which these disadvantages can potentially be overcome. Recombinant techniques allow the production of highly purified allergens. In order to reduce anaphylactic side effects, recombinant allergens can be produced in which IgE binding has been reduced or eliminated. In addition, recombinant techniques allow the production of allergens specifically tailored to the particular patient. Discussions of the use of recombinant allergens in the treatment of Type I allergies can be found in Valenta et al. (Biol. Chem. 380,:815-824, 1999), Ferreira et al. (FASEB J. 12:231-242, 1998), Valenta et al. (Intl. Arch. Allergy Immunol. 116:167-176, 1998), Hsu et al. (U.S. Pat. No. 5,958,891), and Garman et al. (U.S. Pat. No. 5,968,526).

The ability to logically design recombinant allergens for use in immunotherapy, requires an accurate method of determining which allergens an individual responds to, as well as the location and sequence of IgE binding sites (epitopes) within the allergen. Numerous methods of epitope mapping are known in the art. Geysen (U.S. Pat. Nos. 5,194,392 and 5,998,577) describes a method for epitope mapping involving solid support synthesis of short peptides which are then tested for their ability to bind IgE. The amino acid sequence of the of the peptides can be varied and the effect on IgE binding determined. Schramm et al. (J. Immunol. 162:2406-2414, 1999) used synthetic dodecapeptides to map dominant T cell epitopes of the timothy grass pollen allergen pH 1 p 5 b. Site directed mutagenesis outside the regions identified was used to generate point and deletion mutations which were tested for IgE binding by Western blotting. Pandjaitan et al. (Gene, 237:333-342, 1999) studied IgE binding to allergens using a sandwich ELISA assay. The assay utilized recombinant fusion proteins in which the allergen was fused to a birch pollen profilin hexapeptide which was in turn used to anchor the fusion protein to the assay wells via a monoclonal antibody to the profilin hexapeptide. Helm et al. (J. Allergy Clin. Immunol. 105:378-384, 2000) used decapeptides synthesized on membranes to study IgE binding to the soy allergen p34/Gly m Bd 30K. In an earlier study, Helm et al. (Intl. Arch. Allergy Immunol. 117:29-37, 1998) used synthetic 15mer peptides bound to a cellulose membrane to map IgE epitopes on the P34 allergen of soybeans. Ferreira et al. (J. Exp. Med. 183:599-609, 1996) measured IgE binding to various isoforms of the major Birch pollen allergen Bet v 1 to identify low IgE binding isoforms for use in immunotherapy. Chow et al. (Biochem. J 346:423-431, 2000) describes expression of the Asp f 13 allergen from Aspergillus fumigatus as a fusion protein containing a His tag. Chemically and enzymatically cleaved fragments of this protein were then used to map IgE epitopes by immunoblotting. Selo et al. (Clin. Exp. Allergy 29:1055-1063, 1999) used native and tryptic digests of bovine β lactoglobin isolated from milk in an ELISA assay to map IgE binding. Petersen et al. (Clin. Exp. Allergy 28:315-321, 1998) used synthetic decapeptides to map IgE binding to the Ph1 p1 allergen of timothy grass pollen.

### SUMMARY

Among the several aspects of the invention is provided, a method for allergen characterization comprising obtaining a recombinant fusion protein expressed by a host cell, said recombinant fusion protein containing a first amino acid sequence of a known or suspected allergen or allergen fragment fused to a second amino acid sequence native to the host cell expressing the recombinant fusion protein; attaching the recombinant fusion protein to a substrate through the native protein; contacting the recombinant fusion protein attached to the substrate with a biological sample from an individual; and detecting the binding of immunoglobulin E (IgE) molecules in the biological sample to the recombinant fusion protein. In one embodiment, the above procedure is repeated with multiple fusion proteins. These multiple fusion proteins can be different allergens or fragments thereof, or they can be overlapping fragments of a known or suspected allergen.

Another aspect provides a method for allergen characterization comprising obtaining a recombinant fusion protein expressed by an E. coli bacterium, said recombinant fusion protein containing an amino acid sequence of a known or suspected allergen or allergen fragment fused to an amino acid sequence for thioredoxin; attaching the recombinant fusion protein to a substrate by binding the thioredoxin with an antibody attached to the substrate; contacting the recombinant fusion protein attached to the substrate with a biological sample from an individual; and detecting the binding of immunoglobulin E (IgE) molecules in the biological sample to the recombinant fusion protein. In one embodiment, the above procedure is repeated with multiple fusion proteins. These multiple fusion proteins can be different allergens or fragments thereof, or they can be overlapping fragments of a known or suspected allergen.

A further aspect provides a method for determining the sensitivity of an individual to a suspected allergen comprising obtaining a biological sample from the individual and determining the binding of immunoglobulin E (IgE) to said suspected allergen by any of the above methods for allergen characterization.

An additional aspect provides a method for determining the amount of IgE specific for an allergen in a biological sample, comprising obtaining a biological sample from an individual; and determining the binding of IgE to said allergen by any of the above methods for allergen characterization.

Still another aspect provides a method of immunotherapy, comprising obtaining a biological sample from an individual; determining binding of immunoglobulin E in the sample to a series of overlapping fragments of at least one known or suspected allergen by any of the methods of allergen characterization described above; producing mutated forms of the allergen fragments or full length allergens containing the allergen fragments which were determined to bind immunoglobulin E; determining the binding of immunoglobulin E from the individual to the mutant allergen fragments or full length allergens containing mutant allergen fragments; comparing binding of immunoglobulin E to the mutant allergen fragments or full length allergens to unmutated allergen fragments or full length allergens containing the unmutated allergen fragments; and if said mutated allergen fragments or full length allergens have decreased immunoglobulin E binding as compared to the same allergen fragments or full length allergens without the mutations; administering said mutated allergen fragments or full length allergens containing said mutated allergen fragments to said individual. In one embodiment, the mutation is a substitution. In another embodiment, the substitution is a deletion mutation. In still another embodiment, the mutation is an insertion mutation.

Yet another aspect provides a kit comprising a recombinant fusion protein obtained from a host cell, said recombinant fusion protein containing a first amino acid sequence of a known or suspected allergen or allergen fragment fused to a second amino acid sequence native to said host cell and instructions for using said recombinant fusion protein to determine IgE binding to said known or suspected allergen. In one embodiment the recombinant fusion protein is attached to a solid substrate by the native protein. In another embodiment, the kit further comprises buffers, reagents and/or anti IgE antibodies.

### ABBREVIATION AND DEFINITIONS

IgE=immunoglobulin E

IgG—immunoglobulin G

PCR=polymerase chain reaction

HPR=horseradish peroxidase

PBST=phosphate buffered saline containing 0.05% Tween-20

TBST=50 mM Tris, pH 8.0, 150 mM NaCl, 0.05% Tween-20

PVDF=polyvinylidene fluoride

BSA=bovine serum albumin

PBS=137 mM NaCl, 2.7 mM KC, 4.3 mM Na₂HPO₄ 7H₂O, 1.4 mM KH₂PO₄

As used herein “polynucleotide” and “oligonucleotide” are used interchangeably and refer to a polymeric (2 or more monomers) form of nucleotides of any length, either ribonucleotides or deoxyribonucleotides. Although nucleotides are usually joined by phosphodiester linkages, the term also includes polymeric nucleotides containing neutral amide backbone linkages composed of aminoethyl glycine units. This term refers only to the primary structure of the molecule. Thus, this term includes double- and single-stranded DNA and RNA. It also includes known types of modifications, for example, labels, methylation, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.), those containing pendant moieties, such as, for example, proteins (including for e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide. Polynucleotides include both sense and antisense strands.

As used herein “polypeptide”, “protein” and “peptide” are used interchangeably and refer to a polymer of two or more amino acids. Included within the definition are polypeptides containing one or more analogs of an amino acid, including, for example, unnatural amino acids, polypeptides with substituted linkages, as well as modifications known in the art, both naturally occurring and non-naturally occurring.

As used herein, “sequence” means the linear order in which monomers occur in a polymer, for example, the order of amino acids in a polypeptide or the order of nucleotides in a polynucleotide.

As used herein, a “recombinant” is defined either by its method of production or its structure. In reference to its method of production, it refers to the use of recombinant nucleic acid techniques, e.g., involving human intervention in the nucleotide sequence, typically selection or production. Alternatively, it can be a nucleic acid sequence comprising two fragments which are not naturally contiguous or operably linked to each other.

### BRIEF DESCRIPTION OF THE DRAWINGS

These and other features, aspects, and advantages of the present invention will become better understood with regard to the following description, appended claims and accompanying figures where:

FIG. 1 shows the construction of the glycinin fusion fragment proteins. The N-terminal (N) and C-terminal (C) portion of each construct correspond to the 13 kDa thioredoxin fusion partner and the 3 kDa 6xHis tag, respectively. The segments in bold represent soybean glycinin G1 acidic chain sequence (SEQ ID NO: 2) beginning and ending at the indicted residues. The glycinin sequences are arranged to highlight the overlapping segments of primary structure. The figure is not to scale.

FIG. 2 shows an SDS-PAGE gel in which glycinin fusion fragments were separated with a 12.5% separating/5% stacking SDS-PAGE gel and Coomassie stained. The left lane in each gel contains molecular weight standards as indicated. A) lanes were loaded with purified glycinin fusion fragment proteins as indicated, 0.5 μg protein per lane. B) Combined glycinin fusion fragment protein sample, 0.5 μg protein per lane.

FIG. 3 shows immunoblotting of glycinin fusion fragment proteins with pooled soy-sensitive sera. SDS-PAGE was performed with 10-20% Novex pre-cast gels. Transfer to PVDF was performed as described in Example 1 for 1 hour (A) or 2 hours (B and C). Membranes were incubated in diluted sera solution with a total volume of 20 mL (A and B) or 1 mL (C). Antibody dilutions (1:20 pooled soy-sensitive sera, 1:10000 goat anti-human IgE-HRP) and chemiluminescent detection were identical for all three immunoblots.

FIG. 4 shows glycinin FFP-ELISA with soy-sensitive sera (SSS) and nonsensitive sera (NSS). Relative absorbance units were produced by subtracting the absorbance of control wells that received no fusion protein from the absorbance of all other wells for the serum type. Error bars are one standard error of the mean. (*=p<0.001 and ⁺=p<0.01 by student's t-test)

FIG. 5 shows the results of the fusion protein ELISA (FP-ELISA). Relative absorbance units were produced by subtracting the absorbance of control wells that received no fusion protein from the absorbance of all other wells for that serum sample. Each bar represents the average absorbance of three wells. Antigens used were glycinin G1, G2, G3 and G5 acidic chains (g1a, g2a, g3a, and g5a); kunitz soybean trypsin inhibitor (kti); the propeptide form of P34 (pp34); the mature form of P34 (mp34); and thioredoxin control (thio).

FIG. 6 shows immunoblotting of glycinin G1 acidic chain fusion fragment proteins with individual serum samples. Left panel: Molecular weight standards (S) and glycinin G1 acidic chain fusion fragment proteins (P) separated with 12.5% SDS-PAGE and stained with Coomassie Brilliant Blue for reference. Right panel: Immunoblotting of glycinin G1 acidic chain fusion fragment proteins transferred to PVDF and cut into strips with individual serum samples. Serum samples are: BCO (A), C-F (B), R-B (C), SAY (D), JAK (E), DJH (F), BMH (G), CSB (H).

FIG. 7 shows glycinin fusion fragment protein ELISA (FFP-ELISA). Relative absorbance units were produced by subtracting the absorbance of control wells that received no fusion protein from the absorbance of all other wells for that serum type. Schematic representation of the fragments used is shown in FIG. 1.

FIG. 8 shows the aligned sequences of glycinin acidic chains. Sequences from glycinin acidic chains as summarized from data reported by Staswick et al. (J Biol. Chem., 259:13431-13435, 1984) and Neilsen et al. (Plant Cell, 1:313-328, 1989) were aligned for homologous regions. IgE epitopes for pooled soy-allergic sera are highlighted in gray. Aligned sequenced of glycinin G3-G5 acidic chains are only partial sequences. Residues that are unique to that acidic chain are in bold. Conserved residues are indicated by an asterisk. G1 is SEQ ID NO: 2, G2 is SEQ ID NO: 20, G3 is SEQ ID NO: 21, G4 is SEQ ID NO: 22, and G5 is SEQ ID NO: 23.

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications, databases and other references cited in this application are herein incorporated by reference in their entirety as if each individual publication, patent, patent application, database or other reference were specifically and individually indicated to be incorporated by reference.

The present invention measures binding of immunoglobulin E to recombinant fusion proteins containing amino acid sequences of known or suspected allergens. The fusion proteins are characterized by containing one or more sequences encoding the known or suspected allergens fused to an additional amino acid sequence which is native to the host cell in which the fusion protein was produced. In addition, the fusion protein may optionally contain a purification moiety to aid in purification of the recombinant fusion protein from other proteins produced by the host cell.

Methods for the production of fusion proteins are well known in the art and can be found in standard molecular biology references such as Sambrook et al. Molecular Cloning, 2nd ed., Cold Spring Harbor Laboratory Press, 1989 and Ausubel et al., Short Protocols in Molecular Biology, 3rd ed, Wiley and Sons, 1995. In general, a fusion protein is produced by first constructing a fusion gene which is inserted into a suitable expression vector, which is in turn used to transfect a suitable host cell. In general, recombinant fusion constructs are produced by a series of restriction enzyme digestions and ligation reactions which result in the desired sequences being incorporated into a plasmid. If suitable restriction sites are not available, then synthetic oligonucleotide adapters or linkers can be used as is known to those skilled in the art and described in the references cited above. The polynucleotide sequences encoding allergens and native proteins can be assembled prior to insertion into a suitable vector or the sequence encoding the allergen can be inserted adjacent to a sequence encoding a native sequence already present in a vector. Insertion of the sequence within the vector should be in frame so that the sequence can be transcribed into a protein.

It will be apparent to those of ordinary skill in the art, that the precise restriction enzymes, linkers and/or adaptors required as well as the precise reaction conditions will vary with the sequences and cloning vectors used. The assembly of DNA constructs, however, is routine in the art and can be readily accomplished by the skilled technician without undue experimentation.

The polynucleotide sequence may encode the full length amino acid sequence of a known or suspected allergen or it may encode only a fragment of the allergen. Polynucleotides encoding allergen fragments can be obtained by any suitable means known in the art. For example, fragments can be obtained by restriction enzyme digestion of full length coding regions. Care should be taken so that the fragments produced retain the proper reading frame or can be modified so that the resulting fragment is in frame. Alternatively, fragments can be produced by use of the polymerase chain reaction. In this method primers are designed to produce fragments of the allergen. The primers are preferably designed so that the fragments produced will be in frame with the sequence encoding the native protein when inserted into a suitable expression vector. In one embodiment, a series of overlapping fragments are produced which span the length of the known or suspected allergen. In another embodiment these fragments are at least 36 nucleotides in length. In still another embodiment, the fragments overlap by at least 18 nucleotides. In yet another embodiment, the primers are designed for read-through. Methods for the design of primers are well known in the art and can be found for example in Innis et al., PCR Protocols, Academic Press, 1990, Chap. 1, and other references cited herein. In addition, several computer programs are commercially available which can be used to design primers.

It will be recognized by those of skill in the art, that the exact conditions for production of polynucleotide sequences encoding known or suspected allergens or fragments thereof will vary with the composition of the primers and template used. General guidelines for conducting PCR reactions can be found, for example in Innis et al., PCR Protocols, Academic Press, 1990, Ausubel et al., Short Protocols in Molecular Biology, 3rd ed, Wiley and Sons, 1995 and U.S. Pat. Nos. 4,683,195 and 4,683,202. Methods for the optimization of PCR reactions for particular polynucleotide sequences are well known in the art.

Optimization of PCR reaction conditions is routine can be readily accomplished by one of ordinary skill in the art without undue experimentation.

The inclusion of the native protein (fusion partner) within the fusion protein stabilizes expression of polypeptides that might not be expressed efficiently without the presence of the fusion partner. Since the fusion partner is preferably native to the host cell in which the fusion protein is being expressed, the fusion partner provides the fusion protein with some native character to assist in the expression of a wide range of proteins with differing characteristics (e.g. hydrophobic, hydrophillic, acidic, basic, etc.). In addition, the fusion partner, allows for a greater amount of the expressed fusion protein to be in soluble form. This increased solubility is due to the fusion protein having a greater likelihood of properly folding due to the presence of the fusion partner. Proper folding is particularly important in the detection of conformational (discontinuous) epitopes. IgE epitopes can be represented by a linear sequence of adjacent amino acids (continuous epitopes) or by non adjacent sequences that are brought together by folding of the protein (discontinuous or conformational epitopes). Because the presence of the fusion partner increases the possibility of proper folding, the present method is particularly useful for the detection of conformational epitopes. Synthetic peptides used in allergen characterization are not of sufficient length to determine conformational epitopes. Recombinant fusion proteins which do not include a native fusion partner are less likely to properly fold and so are less useful for the detection of conformational epitopes.

In one embodiment, the polynucleotide encoding the fusion protein also contains a sequence encoding a purification moiety, located at either the 3′ or 5′ ends. Such purification moieties can be used to purify and/or isolate the fusion protein by affinity chromatography. Various purification moieties and methods for their use are known in the art. Representative examples can be found in U.S. Pat. Nos. 4,703,004, 4,782,137, 4,845,341, 5,935,824, 5,643,758 and 5,594,115. In one embodiment, the purification moiety comprises a stretch of six histidine residues called a 6x-his-tag. This purification moiety has a high affinity for nickel. A resin with bound nickel ions is used in an affinity chromatography purification step to separate the fusion protein from the protein milieu of the host cell. Non-limiting illustrations of the assembly of DNA constructs encoding fusion proteins useful in the present invention can be found in the examples that follow.

DNA constructs encoding fusion proteins can then be placed into a suitable vector to transform a host cell. The vector can be either a cloning vector or an expression vector. A cloning vector is a self-replicating DNA molecule that serves to transfer a DNA segment into a host cell. The three most common types of cloning vectors are bacterial plasmids, phages, and other viruses. An expression vector is a cloning vector designed so that a coding sequence inserted at a particular site will be transcribed into mRNA. Both cloning and expression vectors contain nucleotide sequences that allow the vectors to replicate in one or more suitable host cells. In cloning vectors, this sequence is generally one that enables the vector to replicate independently of the host cell chromosomes, and also includes either origins of replication or autonomously replicating sequences. Various bacterial and viral origins of replication are well known to those skilled in the art and include, but are not limited to, the pBR322 plasmid origin, the 2 μ plasmid origin, and the SV40, polyoma, adenovirus, VSV and BPV viral origins (Ausubel et al., ed., Short Protocols in Molecular Biology, 3rd ed., Wiley & Sons, 1995).

The polynucleotide constructs of the present invention are used to produce fusion proteins by the use of recombinant expression vectors containing the sequence. Suitable expression vectors include chromosomal, non-chromosomal and synthetic DNA sequences, for example, SV 40 derivatives; bacterial plasmids; phage DNA; baculovirus; yeast plasmids; vectors derived from combinations of plasmids and phage DNA; and viral DNA such as vaccinia, adenovirus, fowl pox virus, retroviruses, and pseudorabies virus. In addition, any other vector that is replicable and viable in the host may be used.

The nucleotide sequence of interest may be inserted into the vector by a variety of methods. In the most common method the sequence is inserted into an appropriate restriction endonuclease site(s) using procedures commonly known to those skilled in the art and detailed in, for example, Sambrook et al., Molecular Cloning, A Laboratory Manual, 2nd ed., Cold Spring Harbor Laboratory Press, (1989) and Ausubel et al., Short Protocols in Molecular Biology, 3rd ed., John Wiley & Sons (1995).

In an expression vector, the sequence of interest is operably linked to a suitable expression control sequence or promoter recognized by the host cell to direct mRNA synthesis. Promoters are untranslated sequences located generally 100 to 1000 base pairs (bp) upstream from the start codon of a structural gene that regulate the transcription and translation of nucleic acid sequences under their control. Promoters are generally classified as either inducible or constitutive. Inducible promoters are promoters that initiate increased levels of transcription from DNA under their control in response to some change in the environment, e.g. the presence or absence of a nutrient or a change in temperature. Constitutive promoters, in contrast, maintain a relatively constant level of transcription. In addition, useful promoters can also confer appropriate cellular and temporal specificity. Such promoters include those that are developmentally-regulated or organelle-, tissue- or cell-specific.

A nucleic acid sequence is operably linked when it is placed into a functional relationship with another nucleic acid sequence. For example, DNA for a presequence or secretory leader is operatively linked to DNA for a polypeptide if it is expressed as a preprotein which participates in the secretion of the polypeptide; a promoter is operably linked to a coding sequence if it affects the transcription of the sequence; or a ribosome binding site is operably linked to a coding sequence if it is positioned so as to facilitate translation. Generally, operably linked sequences are contiguous and, in the case of a secretory leader, contiguous and in reading frame. Linking is achieved by blunt end ligation or ligation at restriction enzyme sites. If suitable restriction sites are not available, then synthetic oligonucleotide adapters or linkers can be used as is known to those skilled in the art (Sambrook et al., Molecular Cloning, A Laboratory Manual, 2nd ed., Cold Spring Harbor Laboratory Press, (1989) and Ausubel et al., Short Protocols in Molecular Biology, 3rd ed., John Wiley & Sons (1995)).

Common promoters used in expression vectors include, but are not limited to, CMV promoter, LTR or SV40 promoter, the E. coli lac or trp promoters, and the phage lambda PL promoter. Other promoters known to control the expression of genes in prokaryotic or eukaryotic cells can be used and are known to those skilled in the art. Expression vectors may also contain a ribosome binding site for translation initiation, and a transcription terminator. The vector may also contain sequences useful for the amplification of gene expression.

Vectors can and usually do contain a selection gene or selection marker. Typically, this gene encodes a protein necessary for the survival or growth of the host cell transformed with the vector. Examples of suitable selection markers include dihydrofolate reductase (DHFR) or neomycin resistance for eukaryotic cells and tetracycline or ampicillin resistance for E. coli. Selection genes in plants include genes that confer resistance to bleomycin, gentamycin, glyphosate, hygromycin, kanamycin, methotrexate, phleomycin, phosphinotricin, spectinomycin, streptomycin, sulfonamide and sulfonylureas. Maliga et al., Methods in Plant Molecular Biology, Cold Spring Harbor Laboratory Press, 1995, p. 39.

In addition, vectors can also contain marker sequences. Suitable markers include, but are not limited to, alkaline phosphatase (AP), myc, hemagglutinin (HA), β-glucuronidase (GUS), luciferase, and green fluorescent protein (GFP).

A further embodiment of the present invention relates to transformed host cells containing constructs of the present invention. The host cell can be a higher eukaryotic cell, such as a plant or animal cell, a lower eukaryotic cell such as a yeast cell, an insect cell or a bacterium. Introduction of the construct into the host cell can be accomplished by a variety of methods including calcium phosphate transfection, DEAE-dextran mediated transfection, Polybrene, protoplast fusion, liposomes, direct microinjection into the nuclei, scrape loading, and electroporation.

Following expression, the fusion proteins are isolated from the host cells. If the fusion protein is secreted, the protein may be isolated from the medium containing the host cells. If the fusion protein is not secreted, the host cells are disrupted, and the fusion protein isolated from the cell lysate. Several methods for the disruption of host cells are known to those of ordinary skill in the art and can be used in the practice of the present invention. These include, but are not limited to, mechanical disruption such as with the use of cell homogenizers, freezing and thawing, enzymatic digestion, sonication, and chemical disruption, such as the use of detergents.

Once the medium has been collected or the cell lysate obtained, the fusion protein is isolated from the unwanted proteins of the host cell and/or medium. Various methods for isolation of proteins from cells and tissue are known to those of skill in the art. These include precipitation by, for example, ammonium sulfate or ethanol precipitation, acid extraction, anion or cation exchange chromatography, phosphocellulose chromatography, hydrophobic interaction chromatography, affinity chromatography, hydroxylapatite chromatography, lectin chromatography, high performance liquid chromatography (HPLC), electrophoresis under native or denaturing conditions, isoelectric focusing, and immunoprecipitation. In one preferred embodiment, fusion proteins are isolated by affinity chromatography using a purification moiety as described above and in the examples that follow.

Once isolated, the recombinant protein is used in an immunoassay to detect binding of immunoglobulin E molecules obtained from an individual suspected of being allergic to the allergen contained in the fusion protein. Various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in vivo immunoassays (using colloidal gold, enzyme or radioisotope labels, for example), western blots, immunoprecipitation reactions, agglutination assays (e.g., gel agglutination assays, hemagglutination assays), complement fixation assays, immunofluorescence assays, and immunoelectrophoresis assays, etc. In one embodiment, IgE is detected by detecting binding of a secondary antibody or reagent to the IgE. In a further embodiment, the secondary antibody is labeled. Any suitable label can be used. Non-limiting examples of suitable labels include radioactive labels, such as radionuclides, fluorophores or fluorochromes, enzymes, vitamins and steroids. Many means are known in the art for detecting binding in an immunoassay and are envisioned for use. As will be readily recognized by those of ordinary skill in the art, the exact means of detection will vary with what, if any, label is used in the immunoassay.

In one embodiment, binding of IgE to fusion proteins is accomplished by an ELISA assay. Fusion proteins are attached to a solid substrate, for example, the wells of multi-well ELISA plates which are available from a variety of commercial vendors. In one embodiment, the fusion proteins are attached to the solid substrate by means of an antibody to the fusion partner (native peptide). For example, the wells of multi-well ELISA plate are coated with an antibody to the fusion partner. Purified fusion proteins are then added in excess so that the fusion partner antibodies are saturated with fusion protein and excess fusion proteins washed away. It is thought that this results in approximately equal amounts of fusion protein being present in each well. In addition, it is thought that this results in an additional purification step for the fusion protein, since proteins which do not bind to the antibody will be removed in the washing step.

Once the fusion protein is bound to the solid substrate, it is brought in contact with a biological sample from an individual suspected of being allergic to the allergen contained in the fusion protein. Any biological fluid containing IgE can be used. Examples include, blood, blood plasma, blood serum, and other body fluids such as tears, nasal and bronchial secretions. In one embodiment, the biological sample is blood serum. If necessary, the biological sample can be diluted prior to use in order to reduce the amount of sample required for each test or to bring the values within an established standard. The sample and the fusion protein are incubated together for a time sufficient to allow interaction between the known or suspected allergen contained in the fusion protein and the IgE contained in the sample. After a suitable time is passed, the IgE bound to the fusion protein is separated from the unbound IgE. Any method capable of separating bound and free IgE can be used. If the fusion protein is bound to a solid substrate, separation can be accomplished by washing away the unbound IgE. If the binding of the IgE and fusion protein occurs without the use of a solid substrate, separation can be accomplished, for example, for precipitation and/or centrifugation.

Once the bound and free IgE are separated, binding of the IgE to the fusion protein is detected. Any method capable of detecting binding of IgE to a protein can be used. In one embodiment, detecting is by contacting the IgE fusion protein complex with a reagent, preferably a second antibody, which specifically binds to IgE. In another embodiment, the reagent has a detectable label. In one preferred embodiment, the reagent is a goat anti-human IgG peroxidase conjugate. As will be apparent to those of skill in the art, the exact method of detection will depend on the label used. For example, if a calorimetric label is used an optical detection method can be used. If a fluorescent label is used, a fluorometric method can be used, and if a radioactive label is used an energy emission detection system such as a scintillation counter can be used. Other methods of detection are known in the art and are considered within the scope of the present invention.

The present invention can be used with fusion proteins containing full length sequences of known or suspected allergens or with fusion proteins containing fragments of known or suspected allergens. In one embodiment, overlapping fragments of a full length allergen are produced and used to detect IgE binding to the fragments using any of the methods described above. Using this method, it is possible to determine, where within an allergen, IgE binds.

It will be apparent to those of ordinary skill in the art, that although the present method has been described in the context of the binding of IgE to an allergen, the method can be modified to determine the binding of other immunoglobulin isotypes (e.g. IgG, IgM, IgA, etc) from individuals.

It is thought that the present invention will have wide application in the area of allergy diagnostics and treatment, especially in the area of food allergies. For example, and without limitation, the present method can be used as a diagnostic tool in the determination of protein-specific food allergies rather than organism-specific food allergies. This type of diagnosis could assist clinicians in counseling patients in allergen avoidance techniques. The present invention can also be used to determine regions of allergenic proteins that do not bind IgE. This information can then be used to produce recombinant mutant proteins that retain the characteristics of the original protein, but are mutated to lessen or eliminate IgE binding. Such mutations can be substitution mutations, insertion mutations or deletion mutations. Mutations can be accomplished by any means known in the art including chemical mutagenesis, enzymatic mutagenesis and oligonucleotide mutagenesis. Methods for introducing mutations are well known in the art and can be found in standard references, such as, Sambrook et al. Molecular Cloning, 2nd ed., Cold Spring Harbor Laboratory Press, 1989;

Ausubel et al., Short Protocols in Molecular Biology, 3rd ed, Wiley and Sons, 1995; and Howe, Gene Cloning and Manipulation, Cambridge University Press, 1995, Chap. 7. These mutant proteins can then be used in a therapeutic manner or to genetically manipulate species so that they produce proteins with decreased binding of IgE. One possible therapeutic use is immunotherapy in which individuals are “vaccinated” with mutant versions of allergens lacking IgE binding sites (epitopes). The present invention can also be applied to screening allergenic responses for a variety of food sources in which genetic sequence information is available. The present invention can be used for the quantitative determination of IgE molecules specific for a particular allergen and for monitoring changes in the specific IgE over time. The present invention is also useful for assessing alterations in IgE binding due to processing, such as food processing, and to identify hypoallergenic proteins, either naturally occurring or produced by mutagenesis.

As discussed above, the present method has therapeutic applications especially in the area of immunological therapies for allergies. Using the method of the present invention, IgE binding sites can be identified. Then using methods of molecular biology, the IgE binding sites can be mutated or eliminated so that the resulting mutated protein has a decreased capacity to bind IgE. Although possessing reduced or no capacity for IgE binding, the mutated protein preferably will bind other immunoglobulins such as IgG. The present invention can also be used to verify IgG binding. Binding of IgG to the mutant allergen can be determined as described herein for IgE binding by the substitution of IgG for IgE and the use of an anti IgG second antibody. Modification of invention for use with IgG can be accomplished by one of ordinary skill in the art without undue experimentation.

Also within the scope of the present invention are kits comprising a recombinant fusion protein obtained from a host cell, said recombinant fusion protein containing a first amino acid sequence of a known or suspected allergen or allergen fragment fused to a second amino acid sequence native to said host cell and instructions for using said recombinant fusion protein to determine IgE binding to said known or suspected allergen. In one embodiment the recombinant fusion protein is attached to a solid substrate by the native protein. Examples of suitable solid substrates include microtiter plates such as those commonly available from commercial sources, membranes and microspheres. In another embodiment, the kit further comprises buffers, reagents and/or anti IgE antibodies. The anti IgE antibodies provided can optionally include label to aid in detection. Reagents can include materials for the detection of labels on the anti IgE antibodies.

### EXAMPLES

The soybean protein glycinin was chosen as a non-limiting example to illustrate the present invention. Soybean glycinin was chosen because of its abundance in the soybean seed, its known involvement in allergic reactions involving IgE binding, and its homology to other legume family storage proteins. It should be noted that the present invention is not limited in its application to glycinin or legume storage proteins, but that the use of glycinin is for illustrative purposes only. Using the guidance provided herein, and knowledge generally available in the art, the skilled technician can adapt the methods of the present invention for use with allergens other than glycinin without undue experimentation.

### Example 1

Identification of IgE Epitopes for the Glycinin G1 Acidic Chain

Methods

PCR Cloning of Glycinin G1 Acidic Chain Fragments:

PCR primers were designed for read-through and to divide the glycinin G1 acidic chain (GenBank accession no: X15121; SEQ ID NO: 1) into overlapping fragments, with one fragment representing the identified IgE-binding region. Primers used (G1BADL, 5′-gccatgggttccagagag (SEQ ID NO: 3); G1IP1, 5′-acaacccggctatatcatgc (SEQ ID NO: 4); G1IP2, 5′-tttctaaaatatcagcaagagcaa (SEQ ID NO: 5); G1IP3, 5′-tgcatgttccaagaattcca (SEQ ID NO: 6); G1IP4, 5′-aaaccacccacggacgag (SEQ ID NO: 7); G1BADR, 5′-gcgtttgctttggcttcct (SEQ ID NO: 8)) were synthesized by Sigma-Genosys (The Woodlands, TX). The primers amplified the regions of the glycinin G1 acidic chain polypeptide (GenBank accession no: X15121; SEQ ID NO: 2) given in Table 1. The PCR reaction utilized Taq polymerase (Gibco-BRL) to leave single 3′-deoxyadenosine overhangs and was run for 30 cycles. PCR products were assessed for purity by agarose gel electrophoresis, and either gel purified or used directly in PCR cloning. PCR cloning was performed using the pBAD/TOPO Thiofusion vector expression system following manufacturer-supplied protocols (Invitrogen, Carlsbad, Calif.). This expression system was chosen for its tight control over basal-level expression, the inclusion of a 6xHis tag for ease of purification, and the ability of the fusion partner (thioredoxin) to stabilize the expression of recombinant protein. Transformed TOP10 E. coli colonies were screened for plasmid insert size and orientation by endonuclease mapping. Constructs with inserts of the correct size and orientation were sequenced to verify the frame of the insert and to check for PCR errors.

TABLE 1

| Fragment | Amino Acid Residues | Primer 1 | Primer 2 |
|---|---|---|---|
| 1 | S22-C107 | G1BADL | G1IP1 |
| 2 | S22-A232 | GIBADL | G1IP3 |
| 3 | S22-K306 | G1BADL | G1BADR |
| 4 | F192-A232 | G1IP2 | G1IP3 |
| 5 | F192-K306 | G1IP2 | G1BADR |
| 6 | K266-K306 | G1IP4 | G1BADR |

Expression and Purification of Glycinin G1 Acidic Chain Fusion-Fragment Proteins:

Overnight cultures grown in Luria broth (LB) containing ampicillin (50 ug/mL) were used to inoculate 500 mL of LB/ampicillin. These 500 mL cultures were grown at 37° C. until the OD₆₀₀ₙₘ reached approximately 0.5, at which time they were induced to a final concentration of 0.02% with L-arabinose. Induced cultures were grown for 3-4 hours before harvesting by centrifugation at 5000×g (10 minutes at 4° C.). Cell pellets from 500 mL cultures were stored at

−20 °C. if not used immediately. Typical expressions of thioredoxin fusion proteins produced protein in both the soluble and insoluble fractions. Some fusion proteins accumulated to a greater extent in the insoluble fraction so all expressed fusion proteins were purified from the insoluble fraction. Cell pellets were resuspended in 40 mL cold lysis buffer (50 mM Tris pH 7.9, 500 mM NaCl, 5 mM imidazole) containing 50 μg/mL lysozyme and incubated on ice 20 minutes. Cell lysates were then sonicated three times for 10 seconds at 50% output to shear DNA (Branson Sonifier 450), and centrifuged at 20,000×g (20 minutes at 4° C.). Pelleted material was resuspended in 5 mL lysis buffer containing 6M Urea and was incubated on ice for 2 hours. Insoluble material was removed by centrifugation at 39,000×g (20 minutes at 4° C.), and supernatant was applied to a two milliliter ProBond resin column (Invitrogen, Carlsbad, Calif.) pre-equilibrated with lysis buffer containing 6M Urea. The bound 6xHis tagged recombinant fusion proteins were washed with step gradients of imidazole (5, 20, 40 mM) and eluted with 300 mM imidazole. Purification of recombinant fusion proteins was monitored by 12.5% separating/5% stacking gel SDS-PAGE to assess purity of the eluted protein. Eluted fractions (˜5 mL) were dialyzed overnight against 500 mL of 50 mM Tris, pH 7.9; concentrated with Centricon-30 or Centricon-10 (Amicon); and analyzed for protein concentration by the BCA assay (Pierce Chemical, Rockford, Ill.). Concentrated fusion protein samples were aliquotted and stored at −20° C.

Soybean Glycinin Fusion Fragment Protein Enzyme-Linked Immunosorbent Assay (FFP-ELISA):

Ninety-six-well microtiter plates were incubated with 100 μL of 10 μg/mL anti-thioredoxin antibody (Sigma Chemical Co., St. Louis, Mo.), in carbonate-bicarbonate buffer pH 9.6, overnight at 4° C. Wells were blocked with 200 μL phosphate-buffered saline with 0.05% Tween-20 (PBST) containing 1% bovine serum albumin (BSA) (blocking buffer) for 30 minutes at 37° C. Recombinant fusion proteins diluted to 10 μg/mL in PBST-BSA were added to wells (100 μL), and incubated for 1 hour at 37° C. The purified fusion partner (thioredoxin) alone and the absence of fusion protein were included as controls for the triplicate assays. Pooled sera (soy-sensitive or non-sensitive) diluted 1:10 in PBST-BSA was added (100 μL) to the wells containing the panel of fusion proteins and controls, and incubated for 2hours at 37° C. Goat anti-human IgE-HRP (Bethyl Labs Montgomery, Tex.) diluted 1:10000 in PBST-BSA was added (100 μL) to all wells and incubated for 1 hour at 37° C. Well washes between each incubation, and after the final conjugate antibody incubation, were performed with a Nunc 8-well immunowasher and PBST. Bound HRP conjugate antibody was detected by incubation with 100 μL of tetramethylbenzidine liquid substrate system containing H₂O₂ (Sigma Chemical Co., St. Louis, Mo.) for 30 minutes at room temperature, and quenched by the addition of 100 μL 1M H₂SO₄. The absorbance of the wells was measured on a Dynex plate reader at 450 nm. The top two rows of each plate were used to produce a standard curve with a human IgE quantitation kit (Bethyl Labs, Montgomery, Tex.), using affinity-purified goat anti-human IgE (100 μL of 10 μg/mL in carbonate-bicarbonate buffer pH 9.6) and human reference IgE (100 μL diluted to 0.1 IU/mL to 5.0 IU/mL in blocking buffer). Washes and detection for the top two rows were identical to those of the other wells.

SDS-PAGE and Immunoblotting:

SDS-PAGE was performed with 12.5% separating/5% stacking gels or 10-20% Tris-glycine gradient gels (Novex, San Diego, Calif.). Transfer to PVDF membranes (Millipore Immobilon-P) was performed in a tank apparatus (Hoefer TE 22) for 2 hours, at 50V, and 14° C. with transfer buffer (10 mM Tris, 192 mM glycine, pH 8.3) containing 20% methanol. Membranes were then manually cut into strips and blocked overnight at room temperature with rocking in TBST (50 mM Tris pH 8.0, 150 mM NaCl, 0.05% Tween-20). After washing 3×2 minutes in TBST, membranes were incubated in pooled soy-sensitive sera or pooled non-sensitive sera (diluted 1:20 in TBST) at room temperature with rocking for 2 hours. Membranes were washed 3×2 minutes in TBST then 3×15 minutes in TBST. Goat anti-human IgE-HRP (Bethyl Labs, Montgomery, Tex.) diluted 1:10000 in TBST was then added and incubated at room temperature with rocking for 1 hour. After washing 3×2 minutes and 3×15 minutes in TBST, chemiluminescent detection was performed with ECL Plus reagents following manufacturer-supplied protocols (Amersham Pharmacia, Piscataway, N.J.). Exposure of membranes to BioMax ML film (Eastman Kodak, Rochester, N.Y.) was carried out for 10 seconds to 2 minutes, and films were developed using an automated developer (Eastman Kodak, Rochester, N.Y.).

Soy Sensitive Sera:

Human serum pooled from seven individuals with convincing clinical histories of soybean allergy was obtained. Characterization of clinical symptoms and radioallergosorbent (RAST) scores for each individual in the pool has been previously reported (Herian et al., Intl. Arch. Allergy Appl. Immunol., 92:193-198, 1990). Briefly, soybean sensitivity was confirmed by skin prick test and RAST scores of 2.3-68x normal binding of IgE. Four patients within this pool also had a history of peanut allergy. Pooled non-sensitive sera was obtained from six non-atopic individuals.

Results

Construction Expression and Purification of Glycinin G1 Acidic Chain Fusion Fragment Proteins:

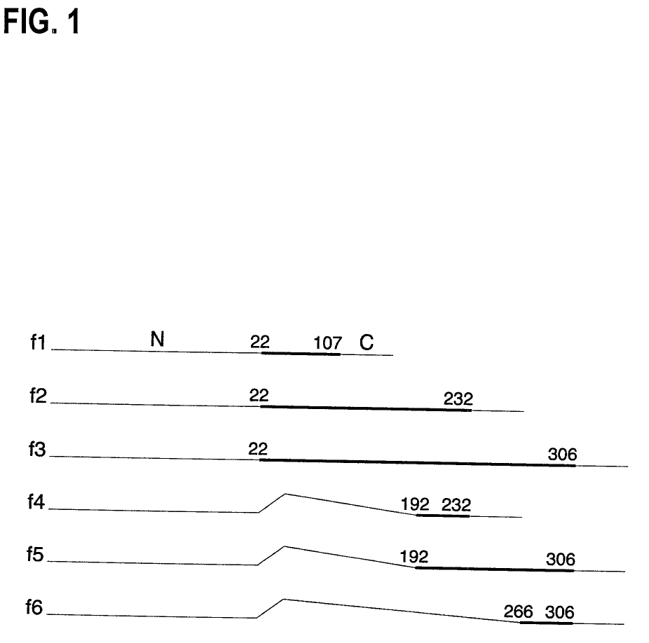

Specific fusion protein fragments of the glycinin G1 acidic chain (SEQ ID NO: 2) were constructed to elucidate IgE-binding domains within a previously identified IgE-binding region (Zeece et al., Food Agric. Immunol., 11:83-90, 1999). A 15 kD proteolytic fragment of glycinin G1 acidic chain, with an N-terminus beginning at residue F192 was shown to bind IgE from pooled soy-sensitive sera by immunoblotting (Zeece et al., Food Agric. Immunol., 11:83-90, 1999). PCR primers were designed to produce fragments covering the entire glycinin G1 acidic chain, with an emphasis on producing overlapping sequence within the IgE-binding fragment as well (FIG. 1). This strategy allowed the search for IgE epitopes to be focused within a limited 15 kD region of the glycinin G1 acidic chain. The fusion proteins f1-f3 begin at residue 22 of the glycinin G1 acidic chain sequence to reflect the mature form of glycinin G1 acidic chain without the signal peptide.

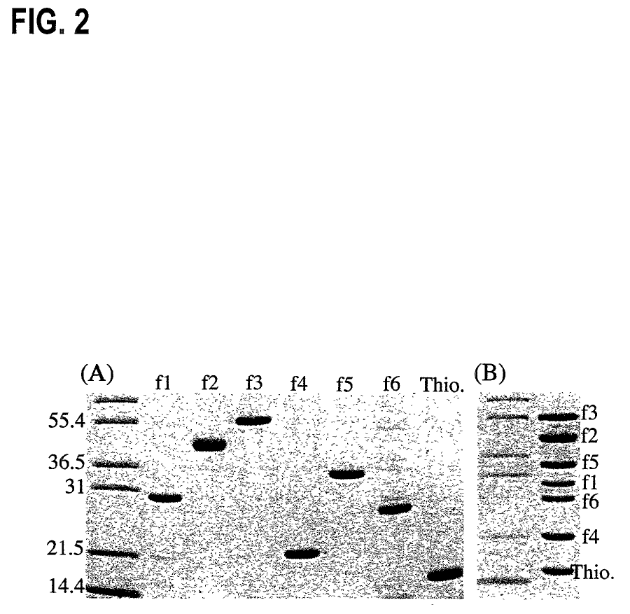

The expression of fusion proteins in E. coli with thioredoxin as the fusion partner was proven to be a very reliable system. Most proteins or protein fragments that were expressed as a thioredoxin fusion were produced at a level of 8-20 mg of purified protein per liter of culture. The purification of the 6xHis tagged fusion proteins was reliably performed by nickel-affinity chromatography. Purity levels were checked by SDS-PAGE and levels of>95% purity were normally attained (FIG. 2a). All of the purified glycinin G1 acidic chain fusion fragment proteins were combined into one sample since their electrophoretic mobility allowed easy resolution with SDS-PAGE (FIG. 2b). This combined sample also aided in the conservation of sera as membrane strips rather than larger membranes could be used in the incubation protocol.

Identification of an IgE-Binding Region by Immunoblotting:

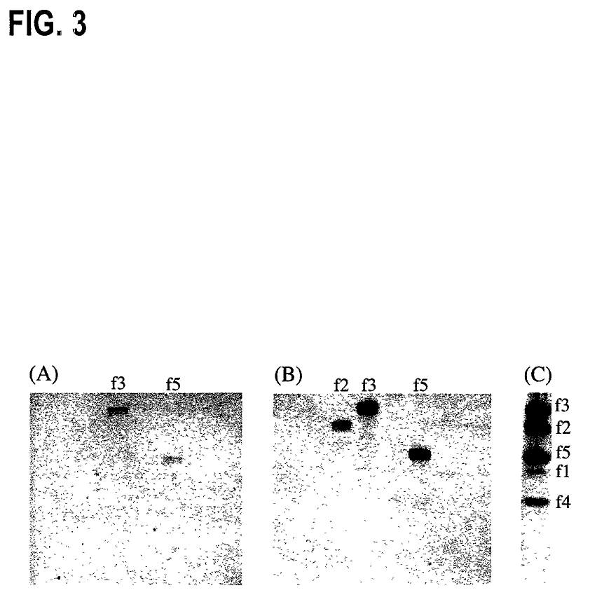

Immunoblotting of the glycinin G1 acidic chain fusion fragment proteins with pooled soy-sensitive sera revealed IgE-binding to multiple fragments (FIG. 3). Variations of the transfer conditions and incubation volumes were performed to optimize the sensitivity of the immunoblotting procedure. Doubling the transfer time from one hour to two hours increased the amount of protein transferred to the membrane as evidenced by less protein staining in the gel after transfer. The increased amount of fusion fragment proteins on the membrane allowed the detection of IgE-binding to f3, f5, and f2 when the less efficient transfer only revealed a response from f3 and f5 (FIGS. 3a and 3b). An increase in sensitivity was also seen with the use of the combined fusion fragment protein sample and incubation of a membrane strip rather than a larger membrane. Immunoblotting of the membrane strip revealed IgE-binding to f3, f5, f2, f4, and a faint response to f1 (FIG. 3c). There was no response of the pooled soy-allergic sera to the thioredoxin fusion partner (FIG. 3) nor of the pooled non-allergic sera to any of the fusion proteins (data not shown).

The pattern of recognition of the fusion fragment proteins indicated that the majority of IgE-binding to glycinin G1 acidic chain was to the region consisting of residues F192-1265 of SEQ ID NO: 2. The absence of any detectable IgE-binding in immunoblotting from f6 excluded residues K266-K306. Truncation of the N-terminal 191 residues of glycinin G1 acidic chain in f5 left a fragment with dominant IgE-binding characteristics, however, the possibility that IgE epitopes exist in the region S22-E191 cannot be ruled out. Indeed, f1 showed some binding of IgE, but the level of response was much lower than the dominant response observed from other fusion fragment proteins. Also, previous results identified a proteolytic fragment corresponding to the N-terminal 191 residues of glycinin G1 acidic chain that did not bind IgE by immunoblotting with this pool of soy-sensitive sera (Zeece et al., Food Agric. Immunol., 11:83-90, 1999). Therefore, the majority of IgE-binding to f2 apparently occurs between residues F192-A232.

Glycinin FFP-ELISA Detection of IgE-Binding:

The fusion proteins used in immunoblotting experiments (f1-f6, thioredoxin) were used in a sandwich-type ELISA to determine IgE-binding. The conditions in the ELISA format do not include a denaturing step such as SDS-PAGE; and therefore, allow the fusion fragment proteins to potentially achieve some native protein folding states and permit the detection of conformational epitopes. Also, the capture antibody used was directed to the fusion partner so that the entire glycinin polypeptide was available to serum antibodies.

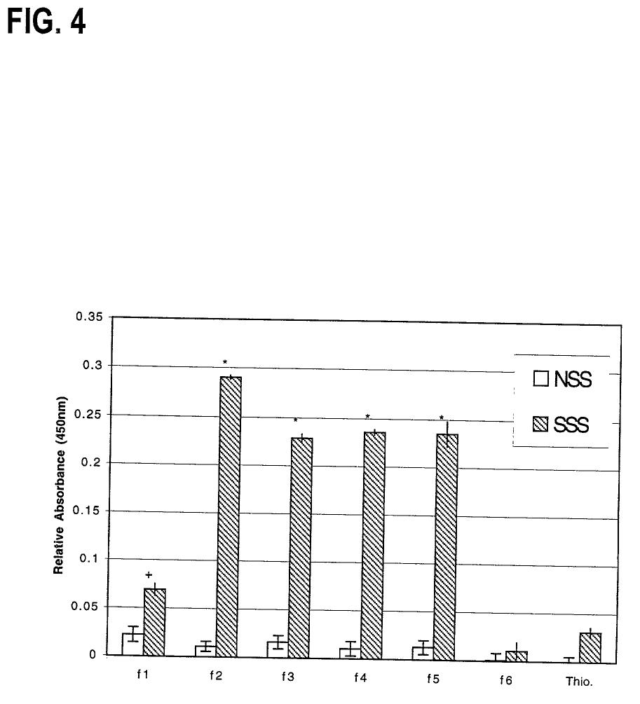

The IgE-binding to the fusion proteins in the FFP-ELISA format showed high responses to f2, f3, f4, and f5 (FIG. 4). However, the relative responses from the fusion proteins was different than in immunoblotting. Fusion protein f4 showed IgE-binding similar to f2, f3, and f5 rather than the weaker response seen in immunoblotting. Similarly, fusion protein f2 showed a response above f3 and f5 when immunoblotting showed the f2 response to be lower than f3 and f5. The binding of IgE to f1 was determined to be significant (p<0.01), giving evidence for IgE epitopes (although minor) in the N-terminal 107 residues of the glycinin G1 acidic chain.

### Example 2

Fusion Protein ELISA for Allergen Characterization

Methods

PCR cloning of Soybean Proteins and Glycinin G1 Acidic Chain Fragments:

PCR primers for use with SEQ ID NO: 1 were designed for read-through of all proteins. Primers used (KTIBADL: 5′-gatttcgtgctcgataatg (SEQ ID NO: 9); KTIBADR: 5′-cagtgattctttatcaagtttttgaa (SEQ ID NO: 10); G5ABADL: 5′-gcccatatgacctccagc (SEQ ID NO: 11); G5ABADR: 5′-ggcatttctagtctgacatcctct (SEQ ID NO: 12); G3ABADL: 5′-gcccatatgagtttcagagagc (SEQ ID NO: 13); G3ABADR: 5′-ggcatttctgctttggctt (SEQ ID NO: 14); G2ABADL: 5′-gcccatatgagagagcaggc (SEQ ID NO: 15); G2ABADR: 5′-5 ggctttgctttggcgttg (SEQ ID NO: 16); MP34L: 5′-gccaagaaaatgaagaaggaa (SEQ ID NO: 17); PROP34L: 5′-catcgttccatattggaccttga (SEQ ID NO: 18); PROP34R: 5′-tcctgcaagaggagagtgat (SEQ ID NO: 19)) were synthesized by Sigma-Genosys (The Woodlands, TX). PCR cloning into the pBAD/TOPO Thiofusion expression vector (Invitrogen, Carlsbad, Calif.) was performed as previously described in Example 1.

Expression and Purification of Fusion Proteins:

The expression and purification of fusion proteins was performed as previously described in Example 1. Briefly, 500 mL cultures were induced with 0.02% L-arabinose for 3-4 hours and harvested by centrifugation. Cell pellets were treated with lysozyme and sonication to lyse the cells and insoluble proteins were pelleted by centrifugation. Analysis by SDS-PAGE of the soluble and insoluble fraction revealed where the majority of the fusion protein was expressed. Fractions containing the majority of the expressed fusion proteins were purified by nickel affinity chromatography using ProBond Resin (Invitrogen, Carlsbad, Calif.). Purified fusion proteins were either concentrated by Centricon-30 (Amicon, Bedford, Mass.) or dialyzed overnight against 2 mM ammonium acetate and dried in a Centrivap Concentrator (Labconco, Kansas City, Mo.). Fusion protein pellets were stored at −70° C. and purified fusion protein solutions were stored at 4° C.

Soybean Fusion Protein Enzyme-Linked Immunosorbent Assay (FP-ELISA):

ELISAs were performed as described previously in Example 1 with the inclusion of additional soy proteins expressed as thioredoxin fusions (glycinin acidic chains from G2 [GenBank X15122], G3 [GenBank X15123], and G5 [GenBank X79467] glycinins; kunitz soybean trypsin inhibitor [GenBank S45092]; propeptide form of P34 [GenBank AB013289]; and mature form of P34 [GenBank AB013289]). The ELISA utilizing only the glycinin G1 acidic chain fragments is referred to as the fusion-fragment protein ELISA (FFP-ELISA) while the ELISA utilizing full-length soy proteins is referred to as the fusion protein ELISA (FP-ELISA).

SDS-PAGE and Immunoblotting:

SDS-PAGE and immunoblotting were performed as previously described in Example 1.

Soy Sensitive Sera:

Individual serum samples from soy-allergic individuals were obtained. Individuals were selected based upon skin prick test to soy extract and clinical histories of soy allergy. Serum samples were also analyzed by Immulite2000 (Diagnostics Products, Los Angeles, Calif.) for total IgE concentration and ImmunoCAP (Pharmacia & Upjohn Diagnostics, Kalamazoo, Mich.) for soy-specific IgE at IBT Laboratories (Lenexa, Kans.).

Results

Soy-Allergic Serum Samples:

Individuals with a clinical history of soy allergy were screened for reactivity by skin prick tests with soy protein extract. Individual serum samples from skin prick test positive individuals were analyzed for total IgE and soy-specific IgE concentrations (Table 2). All individual serum samples displayed elevated IgE levels for their ages. Soy-specific IgE for five of the eight samples was moderate with a score of 2 (0 to 6 scale) and one sample showed high soy-specific IgE with a score of 5.

TABLE 2
Immunlite 2000
Total IgE and ImmunoCAP Soy-specific IgE Determination

| | | | | Total IgE | Soy Specific | Soy Specific |
|---|---|---|---|---|---|---|
| ID # | Patient | Age | Sex | (IU/mL) | (kU/L) | Score |
| 1 | CSB | 21 | F | 363 | 0.79 | 2 |
| 4 | C-F | 5 | M | 557 | 2.55 | 2 |
| 8 | JAK | 21 | M | 99 | 0.11 | 0/1 |
| 9 | SAY | 24 | F | 449 | 1.20 | 2 |
| 10 | BMH | 18 | F | 889 | 2.56 | 2 |
| 18 | DJH | 22 | M | 281 | 0.27 | 0/1 |
| 23 | BDO | 7 | F | 1573 | 54.10 | 5 |
| 24 | R-B | 13 | M | 232 | 2.92 | 2 |

FP-ELISA:

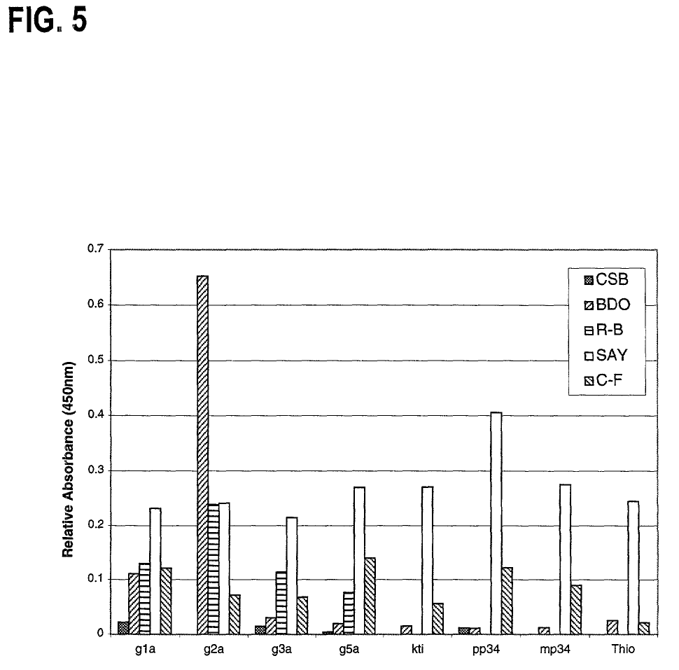

The FP-ELISA test employing full-length soy proteins revealed the IgE-binding preferences of individual serum samples (FIG. 5). Serum samples BDO and R-B showed the strongest IgE-binding to glycinin G2 acidic chain with a lower response to other glycinin acidic chains. Sample C-F displayed moderate IgE-binding to all proteins tested which may indicate a non-specific binding pattern. Sample SAY also displayed IgE-binding to all proteins tested, but this included a high background response to the thioredoxin fusion partner not present in the other samples. The only protein showing a significant response above the SAY thioredoxin IgE-binding was the propeptide form of P34. Since the mature form of P34 did not display IgE-binding, this suggests that SAY responds to the N-terminal propeptide region of P34. Serum sample CSB did not show IgE-binding to any protein in the panel indicating that this individual may be allergic to another soy protein.

Immunoblotting of Glycinin G1 Acidic Chain Fusion Fragment Proteins with Individual Serum Samples:

The response of two individual serum samples in the FP-ELISA to the glycinin G1 acidic chain was lower than the response to the glycinin G2 acidic chain (FIG. 5). However, past experience with glycinin G1 acidic chain showed its higher IgE-binding with a serum pool of soy-allergic individuals which prompted the construction of overlapping fragments for use in this ELISA process. The existing glycinin G1 acidic chain fragments were used with these individual serum samples out of convenience and to test the sensitivity of the FFP-ELISA.

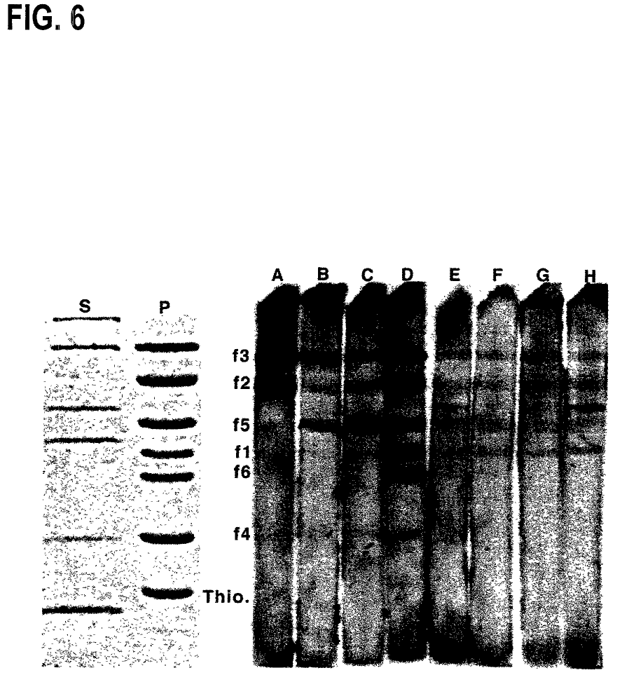

Immunoblotting of the glycinin G1 acidic chain fragments with the individual serum samples was performed first to test for IgE-binding. Four of the individual serum samples displayed significant IgE-binding to multiple fusion fragment proteins (FIG. 6). Sample BDO displayed a strong response to f3 and f2 (FIG. 6, lane A). Samples C-F and R-B showed the strongest IgE-binding to f3 and f5 with lesser responses to f2, f1, and f4 (FIG. 6, lanes B-C). Sample SAY showed strong IgE binding to all of the fusion fragment proteins except for a light response to f4 (FIG. 6, lane D). SAY did not show the background binding to thioredoxin in immunoblotting that was displayed in the FP-ELISA (FIG. 5). This difference may be the result of less efficient thioredoxin binding to the PVDF membrane because of its small size. The four other serum samples showed only background binding to the glycinin G1 acidic chain fragments (FIG. 6, lanes E-H).

FFP-ELISA:

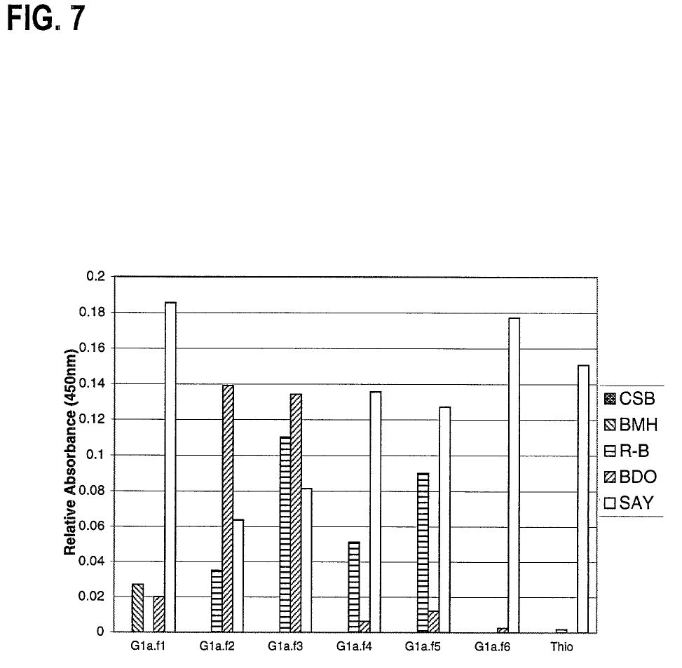

Fusion fragment proteins representing overlapping regions of the glycinin G1 acidic chain were used in the ELISA format to isolate IgE-binding regions for the individual serum samples. Two serum samples displayed significant IgE-binding to multiple fragments (FIG. 7). Samples BDO and R-B showed IgE-binding profiles similar to their responses in immunoblotting. BDO showed strong IgE-binding to f2 and f3 while R-B displayed IgE-binding to f3 and f5 with a lower response to f4 and f2. Once again, serum sample SAY displayed binding to thioredoxin in the ELISA format, but did not show any significant binding to glycinin G1 acidic chain fragments. The other two samples tested, CSB and BMH, did not show any strong binding to the glycinin G1 acidic chain fragments. This was consistent with immunoblotting and FP-ELISA results for these samples. Serum sample C-F was not utilized with this ELISA test because of a shortage of this serum sample.

Immunoblotting of the glycinin G1 acidic chain fusion fragment proteins with individual serum samples showed that four samples had significant IgE-binding to two or more of the fragments (FIG. 6). The FFP-ELISA performed with the same individual serum samples and glycinin G1 acidic chain fusion fragment proteins confirmed the immunoblotting results for two samples (FIG. 7, BDO and R-B) and exposed another sample as binding to the fusion partner thioredoxin (FIG. 7, SAY).

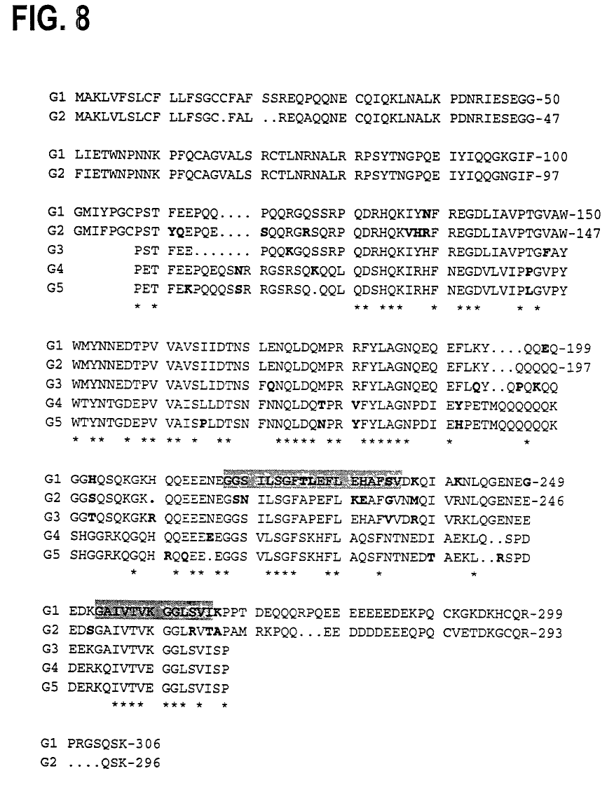

Information from immunoblotting and ELISA tests with the glycinin G1 acidic chain fusion fragment proteins indicated that the IgE-binding regions for BDO and for R-B are residues P108-E191 and F192-1265, respectively. The IgE-binding region of R-B was identical to the region containing IgE epitopes for the pooled soy-allergic sera but the BDO IgE-binding region was unique. Comparing the aligned sequences of glycinin acidic chains in these regions gives an explanation for the broad glycinin response of R-B in the FP-ELISA and the response of BDO to only G1 and G2 glycinin acidic chains (FIG. 8). The IgE-binding regions for the individual serum samples contain many residues that are conserved among all soybean glycinin acidic chains and the G1 and G2 acidic chains display high sequence homology throughout their sequence. This could lead to cross-reactivity in many types of in vitro tests. However, individual amino acid substitutions are sufficient for IgE specificity (Helm et al., J, Allergy Clin. Immunol. 105:378-384, 2000), and a number of residues highlighted in bold are unique to their respective glycinin acidic chain (FIG. 8). Glycinin G2 acidic chain possesses the greatest number of these unique residues (18) which may account for the IgE response above the other acidic chains seen in the FP-ELISA (FIG. 5).

These results illustrate the unique nature of the IgE response of individuals who are allergic to the same food source. This individualized response was seen even with proteins that are as conserved in sequence as the soybean glycinin acidic chains. Continued use of purified recombinant proteins coupled with in depth structural studies would provide further information about the unique character of protein allergens and how they may come to sensitize allergic individuals.

### CONCLUSION

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventors do not intend to be bound by those conclusions and functions, but put them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A method for allergen characterization comprising:
a) obtaining a recombinant fusion protein expressed by a host cell, said recombinant fusion protein containing a first amino acid sequence of a known or suspected allergen or allergen fragment fused to a second amino acid sequence native to the host cell expressing said recombinant fusion protein;
b) attaching said recombinant fusion protein to a substrate through said native protein;
c) contacting said recombinant fusion protein attached to said substrate with a biological sample from an individual; and
d) detecting the binding of immunoglobulin E molecules in said biological sample to said recombinant fusion protein.

2. The method of claim 1, further comprising repeating a) through d) for multiple fusion proteins.

3. The method of claim 2, wherein said multiple fusion fragments are overlapping fragments of a known or suspected allergen.

4. The method of claim 1, wherein said biological sample is selected from the group consisting of blood serum, blood plasma, and mucus.

5. The method of claim 1, wherein said attachment of said recombinant fusion protein to said substrate is accomplished by binding of said native protein to an antibody attached to said substrate.

6. The method of claim 5, wherein said antibody is a polyclonal antibody or a monoclonal antibody.

7. The method of claim 1, wherein said host cell is a bacterium.

8. The method of claim 7, wherein said bacterium is an E coli.

9. The method of claim 1 wherein said native protein is thioredoxin.

10. A method for determining the sensitivity of an individual to a suspected allergen comprising:
a) obtaining a biological sample from said individual; and
b) determining the binding of immunoglobulin E in said biological sample to said suspected allergen by the method of claim 1.

11. The method of claim 10, wherein said biological sample is selected from the group consisting of blood serum, blood plasma, and mucus.

12. A method for determining the amount of immunoglobulin E specific for an allergen in a biological sample, comprising
a) obtaining a biological sample from an individual; and
b) determining the binding of immunoglobulin E to said allergen by the method of claim 1.

13. A method of immunotherapy, comprising:
a) obtaining a biological sample from an individual;
b) determining binding of immunoglobulin E in said sample to a series of overlapping fragments of at least one known or suspected allergen by the method of claim 1;
c) producing mutated forms of the allergen fragments or full length allergens containing the allergen fragments which were determined in (b) to bind immunoglobulin E;
d) determining the binding of immunoglobulin E from the individual of (a) to said mutant allergen fragments of (c) or full length allergens containing said mutant allergen fragments of (c);
e) comparing binding of immunoglobulin E to the allergen fragments or full length allergens of (c) to the unmutated allergen fragments of (b) or full length allergens containing the unmutated allergen fragments of (b); and
f) if said mutated allergen fragments or full length allergens of (c) have decreased immunoglobulin E binding as compared to the same allergen fragments of (b) or full length allergens;
g) administering said mutated allergen fragments or full length allergens containing said mutated allergen fragments to said individual.

14. The method of claim 13, wherein said mutation is a substitution mutation, an insertion mutation or a deletion mutation.

15. A method for allergen characterization comprising:
a) obtaining a recombinant fusion protein expressed by an E coli bacterium, said recombinant fusion protein containing a first amino acid sequence of a known or suspected allergen or allergen fragment fused to a second amino acid sequence of thioredoxin;
b) attaching said recombinant fusion protein to a substrate by binding said thioredoxin with an antibody attached to said substrate;
c) contacting said recombinant fusion protein attached to said substrate with a biological sample from an individual; and
d) detecting the binding of immunoglobulin E molecules in said biological sample to said recombinant fusion protein.

16. The method of claim 15, further comprising repeating a) through d) for multiple fusion proteins.

17. The method of claim 16, wherein said multiple fusion fragments are overlapping fragments of a full length known or suspected allergen.

18. The method of claim 15, wherein said biological sample is selected from the group consisting of blood serum, blood plasma, and mucus.

19. The method of claim 15, wherein said antibody is a polyclonal antibody or a monoclonal antibody.

20. A method for determining the sensitivity of an individual to a suspected allergen comprising:
a) obtaining a biological sample from said individual; and
b) determining the binding of immunoglobulin E in said biological sample to said suspected allergen by the method of claim 15.

21. A method for determining the amount of immunoglobulin E specific for an allergen in a biological sample, comprising
a) obtaining a biological sample from an individual; and
b) determining the binding of immunoglobulin E to said allergen by the method of claim 15.

22. A method of immunotherapy, comprising:
a) obtaining a biological sample from an individual;
b) determining binding of immunoglobulin E in said sample to a series of overlapping fragments of at least one known or suspected allergen by the method of claim 15;
c) producing mutated forms of the allergen fragments or full length allergens containing the allergen fragments which were determined in (b) to binding immunoglobulin E;
d) determining the binding of immunoglobulin E from the individual of (a) to said mutant allergen fragments or full length allergens containing said mutant allergen fragments of (c);
e) comparing binding of immunoglobulin E to the allergen fragments or full length allergens of (c) to the unmutated allergen fragments of (b) or full length allergens containing the unmutated allergen fragments of (b); and
f) if said mutated allergen fragments or full length allergens of (c) have decreased immunoglobulin E binding as compared to the same allergen fragments of (b) or full length allergens;
g) administering said mutated allergen fragments or full length allergens containing said mutated allergen fragments to said individual.

23. A kit comprising a recombinant fusion protein obtained from a host cell, said recombinant fusion protein containing a first amino acid sequence of a known or suspected allergen or allergen fragment fused to a second amino acid sequence native to said host cell said recombinant fusion protein bound to a solid substrate through said native amino acid sequence; and instructions for using said recombinant fusion protein to determine IgE binding to said known or suspected allergen.

24. The kit of claim 23 further comprising an anti IgE antibody.

25. A method for epitope determinationcomprising:
a) obtaining a recombinant fusion protein expressed by a host cell, said recombinant fusion protein containing a first amino acid sequence comprising a known or suspected epitope fused to a second amino acid sequence native to the host cell expressing said recombinant fusion protein;
b) attaching said recombinant fusion protein to a substrate through said native protein;
c) contacting said recombinant fusion protein attached to said substrate with a biological sample containing an immunoglobulin; and
d) detecting the binding of immunoglobulin molecules in said biological sample to said recombinant fusion protein.

26. The method of claim 25 wherein said immunoglobulins are selected from the group consisting of IgA, IgE, IgG, and IgM.


SEQUENCE LISTING (23 sequences)

SEQ ID NO: 1 - DNA, 3527 bp, Glycine max
  TATA_signal (607)..(612)
  exon (639)..(976)
  Intron (977)..(1204)
  exon (1205)..(1458)
  Intron (1459)..(1749)
  exon (1750)..(2307)
  Intron (2308)..(2688)
  exon (2689)..(3282)
  polyA_signal (3136)..(3141)
  polyA_signal (3250)..(3255)
  precursor_RNA (639)..(3282)
  CDS (691)..(976)
  CDS (1205)..(1458)
  CDS (1750)..(2307)
  CDS (2689)..(3075)
  misc_feature (211)..(219): note=“caca box”
  misc_feature (386)..(393): note=“catgcatg element”
  misc_feature (522)..(549): note=“legumin box”
  misc_feature (530)..(537): note=“catgcatg element”
tagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgttgtccctcattcaccttcctctcttccctataaataaccacgcctcaggttctccgcttcacaactcaaacattctcctccattggtccttaaacactcatcagtcatcaccatggccaagctagttttttccctttgttttctgcttttcagtggctgctgcttcgctttcagttccagagagcagcctcagcaaaacgagtgccagatccaaaaactcaatgccctcaaaccggataaccgtatagagtcagaaggagggctcattgagacatggaaccctaacaacaagccattccagtgtgccggtgttgccctctctcgctgcaccctcaaccgcaacgcccttcgtagaccttcctacaccaacggtccccaggaaatctacatccaacaaggtccatcttgtccaaacttcacatataaatatataatagacttaaatatgtttaagggtttgataaatgagggaattttattttagatttttaataattttttttgttttgagtttttatatattaaaatttttgttttgatttcttccatatgacgtaacataatcatatcattgataatgttgggttcctaatttttgtttgtttgttgttttgtaatatgaataggtaagggtatttttggcatgatatacccgggttgtcctagcacatttgaagagcctcaacaacctcaacaaagaggacaaagcagcagaccacaagaccgtcaccagaagatctataacttcagagagggtgatttgatcgcagtgcctactggtgttgcatggtggatgtacaacaatgaagacactcctgttgttgccgtttctattattgacaccaacagcttggagaaccagctcgaccagatgcctagggtgagccacatagcaatattagatattataattctttaaaggtttaaatatcattttagttcgtggagttgcactttctaatttagtacctatagattaaaatatgccaattgaatccttatagttgtgtttttttatccaatttggttcttgtcttgaaataaatggacaatattgtagctgataaaaaaaggaaactggactacattgtaacgttaagattagaattcttaagttctaatactagctggttacagattgacaactatttgttttgacaattcttggcagagattctatcttgctgggaaccaagagcaagagtttctaaaatatcagcaagagcaaggaggtcatcaaagccagaaaggaaagcatcagcaagaagaagaaaacgaaggaggcagcatattgagtggcttcaccctggaattcttggaacatgcattcagcgtggacaagcagatagcgaaaaacctacaaggagagaacgaaggggaagacaagggagccattgtgacagtgaaaggaggtctgagcgtgataaaaccacccacggacgagcagcaacaaagaccccaggaagaggaagaagaagaagaggatgagaagccacagtgcaagggtaaagacaaacactgccaacgcccccgaggaagccaaagcaaaagcagaagaaatggcattgacgagaccatatgcaccatgagacttcgccacaacattggccagacttcatcacctgacatctacaaccctcaagccggtagcgtcacaaccgccaccagccttgacttcccagccctctcgtggctcagactcagtgctgagtttggatctctccgcaaggtacgtacatcattcatcaaagatcaacatacatttatacattaaactaatatttgttgccaaatatttattaattttattgataattaatttttttagaaaatttgtttgatcacttttaatggagtctttcatcttaattacattatttatacttagactaatgatttattgattaataataatcttagatacactataaaatgtgtgacggagttatcttaacacttgcatggattctatcttttctgtctttatatatagaaatagagagaaaaaaaaagaaaagattgatgaaaaaagcaaaacaaaaaatagtattattataaaaatattggatgaatttgttgtgactcttgcatgcattgatgtacgatgcagaatgcaatgttcgtgccacactacaacctgaacgcgaacagcataatatacgcattgaatggacgggcattgatacaagtggtgaattgcaacggtgagagagtgtttgatggagagctgcaagagggacgggtgctgatcgtgccacaaaactttgtggtggctgcaagatcacagagtgacaacttcgagtatgtgtcattcaagaccaatgatacacccatgatcggcactcttgcaggggcaaactcattgttgaacgcattaccagaggaagtgattcagcacactttcaacctaaaaagccagcaggccaggcagataaagaacaacaaccctttcaagttcctggttccacctcaggagtctcagaagagagctgtggcttagagccctttttgtatgtgctaccccacttttgtctttttggcaatagtgctagcaaccaataaataataataataataatgaataagaaaacaaaggctttagcttgccttttgttcactgtaaaataataatgtaagtactctctataatgagtcacgaaacttttgcgggaataaaaggagaaattccaatgagttttctgtcaaatcttcttttgtctctctctctctctcttttttttttctttcttctgagcttcttgcaaaacaaaaggcaaacaataacgattggtccaatgatagttagcttgatcgatgatatctttaggaagtgttggcaggacaggacatgatgtagaagactaaaattgaaagtattgcagacccaatagttgaagattaactttaagaatgaagacgtcttatcaggttcttcatgacttggagctc

SEQ ID NO: 2 - protein, 495 aa, Glycine max
  misc_feature (211)..(219): note=“caca box”
  misc_feature (386)..(393): note=“catgcatg element”
  misc_feature (522)..(549): note=“legumin box”
  misc_feature (530)..(537): note=“catgcatg element”
MAKLVFSLCFLLFSGCCFAFSSREQPQQNECQIQKLNALKPDNRIESEGGLIETWNPNNKPFQCAGVALSRCTLNRNALRRPSYTNGPQEIYIQQGKGIFGMIYPGCPSTFEEPQQPQQRGQSSRPQDRHQKIYNFREGDLIAVPTGVAWWMYNNEDTPVVAVSIIDTNSLENQLDQMPRRFYLAGNQEQEFLKYQQEQGGHQSQKGKHQQEEENEGGSILSGFTLEFLEHAFSVDKQIAKNLQGENEGEDKGAIVTVKGGLSVIKPPTDEQQQRPQEEEEEEEDEKPQCKGKDKHCQRPRGSQSKSRRNGIDETICTMRLRHNIGQTSSPDIYNPQAGSVTTATSLDFPALSWLRLSAEFGSLRKNAMFVPHYNLNANSIIYALNGRALIQVVNCNGERVFDGELQEGRVLIVPQNFVVAARSQSDNFEYVSFKTNDTPMIGTLAGANSLLNALPEEVIQHTFNLKSQQARQIKNNNPFKFLVPPQESQKRAVA

SEQ ID NO: 3 - DNA, 18 bp, Glycine max
gccatgggttccagagag

SEQ ID NO: 4 - DNA, 20 bp, Glycine max
acaacccggctatatcatgc

SEQ ID NO: 5 - DNA, 24 bp, Glycine max
tttctaaaatatcagcaagagcaa

SEQ ID NO: 6 - DNA, 20 bp, Glycine max
tgcatgttccaagaattcca

SEQ ID NO: 7 - DNA, 18 bp, Glycine max
aaaccacccacggacgag

SEQ ID NO: 8 - DNA, 19 bp, Glycine max
gcgtttgctttggcttcct

SEQ ID NO: 9 - DNA, 19 bp, Glycine max
gatttcgtgctcgataatg

SEQ ID NO: 10 - DNA, 26 bp, Glycine max
cagtgattctttatcaagtttttgaa

SEQ ID NO: 11 - DNA, 18 bp, Glycine max
gcccatatgacctccagc

SEQ ID NO: 12 - DNA, 24 bp, Glycine max
ggcatttctagtctgacatcctct

SEQ ID NO: 13 - DNA, 22 bp, Glycine max
gcccatatgagtttcagagagc

SEQ ID NO: 14 - DNA, 19 bp, Glycine max
ggcatttctgctttggctt

SEQ ID NO: 15 - DNA, 20 bp, Glycine max
gcccatatgagagagcaggc

SEQ ID NO: 16 - DNA, 18 bp, Glycine max
ggctttgctttggcgttg

SEQ ID NO: 17 - DNA, 21 bp, Glycine max
gccaagaaaatgaagaaggaa

SEQ ID NO: 18 - DNA, 23 bp, Glycine max
catcgttccatattggaccttga

SEQ ID NO: 19 - DNA, 20 bp, Glycine max
tcctgcaagaggagagtgat

SEQ ID NO: 20 - protein, 485 aa, Glycine max
MAKLVLSLCFLLFSGCFALREQAQQNECQIQKLNALKPDNRIESEGGFIETWNPNNKPFQCAGVALSRCTLNRNALRRPSYTNGPQEIYIQQGNGIFGMIFPGCPSTYQEPQESQQRGRSQRPQDRHQKVHRFREGDLIAVPTGVAWWMYNNEDTPVVAVSIIDTNSLENQLDQMPRRFYLAGNQEQEFLKYQQQQQGGSQSQKGKQQEEENEGSNILSGFAPEFLKEAFGVNMQIVRNLQGENEEEDSGAIVTVKGGLRVTAPAMRKPQQEEDDDDEEEQPQCVETDKGCQRQSKRSRNGIDETICTMRLRQNIGQNSSPDIYNPQAGSITTATSLDFPALWLLKLSAQYGSLRKNAMFVPHYTLNANSIIYALNGRALVQVVNCNGERVFDGELQEGGVLIVPQNFAVAAKSQSDNFEYVSFKTNDRPSIGNLAGANSLLNALPEEVIQHTFNLKSQQARQVKNNNPFSFLVPPQESQRRAVA

SEQ ID NO: 21 - protein, 481 aa, Glycine max
MAKLVLSLCFLLFSGCCFAFSFREQPQQNECQIQRLNALKPDNRIESEGGFIETWNPNNKPFQCAGVALSRCTLNRNALRRPSYTNAPQEIYIQQGSGIFGMIFPGCPSTFEEPQQKGQSSRPQDRHQKIYHFREGDLIAVPTGFAYWMYNNEDTPVVAVSLIDTNSFQNQLDQMPRRFYLAGNQEQEFLQYQPQKQQGGTQSQKGKRQQEEENEGGSILSGFAPEFLEHAFVVDRQIVRKLQGENEEEEKGAIVTVKGGLSVISPPTEEQQQRPEEEEKPDCDEKDKHCQSQSRNGIDETICTMRLRHNIGQTSSPDIFNPQAGSITTATSLDFPALSWLKLSAQFGSLRKNAMFVPHYNLNANSIIYALNGRALVQVVNCNGERVFDGELQEGQVLIVPQNFAVAARSQSDNFEYVSFKTNDRPSIGNLAGANSLLNALPEEVIQQTFNLRRQQARQVKNNNPFSFLVPPKESQRRVVA

SEQ ID NO: 22 - protein, 562 aa, Glycine max
MGKPFTLSLSSLCLLLLSSACFAISSSKLNECQLNNLNALEPDHRVESEGGLIQTWNSQHPELKCAGVTVSKLTLNRNGLHSPSYSPYPRMIIIAQGKGALGVAIPGCPETFEEPQEQSNRRGSRSQKQQLQDSHQKIRHFNEGDVLVIPPSVPYWTYNTGDEPVVAISLLDTSNFNNQLDQTPRVFYLAGNPDIEYPETMQQQQQQKSHGGRKQGQHQQEEEEEGGSVLSGFSKHFLAQSFNTNEDIAEKLESPDDERKQIVTVEGGLSVISPKWQEQQDEDEDEDEDDEDEQIPSHPPRRPSHGKREQDEDEDEDEDKPRPSRPSQGKRNKTGQDEDEDEDEDQPRKSREWRSKKTQPRRPRQEEPRERGCETRNGVEENICTLKLHENIARPSRADFYNPKAGRISTLNSLTLPALRQFQLSAQYVVLYKNGIYSPHWNLNANSVIYVTRGQGKVRVVNCQGNAVFDGELRRGQLLVVPQNFVVAEQAGEQGFEYIVFKTHHNAVTSYLKDVFRAIPSEVLAHSYNLRQSQVSELKYEGNWGPLVNPESQQGSPRVKVA

SEQ ID NO: 23 - protein, 517 aa, Glycine max
MGKPFFTLSLSSLCLLLLSSACFAITSSKFNECQLNNLNALEPDHRVESEGGLIETWNSQHPELQCAGVTVSKRTLNRNGLHLPSYSPYPQMIIVVQGKGAIGFAFPGCPETFEKPQQQSSRRGSRSQQQLQDSHQKIRHFNEGDVLVIPPGVPYWTYNTGDEPVVAISLLDTSNFNNQLDQNPRVFYLAGNPDIEHPETMQQQQQQKSHGGRKQGQHQQQEEEGGSVLSGFSKHFLAQSFNTNEDTAEKLRSPDDERKQIVTVEGGLSVISPKWQEQEDEDEDEDEEYEQTPSYPPRRPSHGKHEDDEDEDEEEDQPRPDHPPQRPSRPEQQEPRGRGCQTRNGVEENICTMKLHENIARPSRADFYNPKAGRISTLNSLTLPALRQFGLSAQYVVLYRNGIYSPHWNLNANSVIYVTRGKGRVRVVNCQGNAVFDGELRRGQLLVVPQNFVVAEQGGEQGLEYVVFKTHHNAVSSYIKDVFRAIPSEVLSNSYNLGQSQVRQLKYQGNSGPLVNP